(19) United States
(12) Patent Application Publication
(10) Pub. No.: US 2023/0085863 A1
(43) Pub. Date: Mar. 23, 2023

(54) TELOMERE LENGTH MODULATION USING FIBROBLASTS

(71) Applicant: FIGENE, LLC, Houston, TX (US)

(72) Inventors: Thomas ICHIM, San Diego, CA (US); Pete O'HEERON, Houston, TX (US)

(21) Appl. No.: 17/904,180

(22) PCT Filed: Feb. 16, 2021
(86) PCT No.: PCT/US2021/018160

§ 371 Date: Aug. 12, 2022

Related U.S. Application Data
(60) Provisional application No. 62/977,604, filed on Feb. 17, 2020.

(51) Int. Cl. A61K 35/33; A61K 35/28; A61K 35/51; A61K 45/06

(52) U.S. Cl. CPC A61K 35/33; A61K 35/28; A61K 35/51; A61K 45/06

(57) ABSTRACT

Disclosed herein are methods and compositions for modulation of telomere length in cells. Aspects are directed to reduction in telomere shortening rate, stabilization of telomere shortening, and/or telomere elongation using fibroblasts or fibroblast-derived products. In some cases, provided are methods for treatment of telomere-associated conditions using fibroblasts or fibroblast-derived products. Fibroblasts may be provided to modulate telomere length in cells of a subject, thereby treating a telomere-associated condition such as, for example, cancer, aging, or idiopathic pulmonary fibrosis.

This application claims priority to U.S. Provisional Pat. Application Serial No. 62/977604, filed Feb. 17, 2020, which is incorporated by reference herein in its entirety.

### TECHNICAL FIELD

Embodiments of the disclosure encompass at least the fields of cell biology, molecular biology, and medicine.

### BACKGROUND

Telomeres are specialized nucleoproteic structures that cap and protect chromosomes from DNA degradation and end-fusion which can lead to chromosomal breaks and recombination [1]. In telomerase-negative cells, each time a cell divides, the telomeres become shorter. When the telomeres get too short, the cell can no longer divide and ultimately leads to cellular senescence and death [2]. Although telomeric sequences vary between species, their essential features are similar between eukaryotes. The telomeric DNA is generally composed of tandem repeats of a basic sequence unit [3].

Telomeric repeats of some organisms are perfect repeats, such as sequence TTAGGG seen in humans or slime mold [4]. Others, such as those of yeast or protozoans, have irregular repeat sequences. In general, the G rich strand runs 5’ to 3’ to the chromosomal terminus. The length of the repeat sequences range from a few to tens of kilo base pairs [5].

Generally, the DNA replicative machinery acts in the 5’ to 3’ direction, and synthesis of the lagging strand occurs discontinuously by use of short RNA primers that are degraded following strand synthesis. Since sequences at the 3’ end of a linear DNA are not available to complete synthesis of the region previously occupied by the RNA primer, the terminal 3’ region of the linear chromosome is not replicated. This “end replication problem” is solved by the action of telomerase, a telomere-specific ribonucleoprotein reverse transcriptase. The telomerase enzyme has an integral RNA component that acts as a template for extending the 3’ end of the telomere. Repeated extensions by telomerase activity results in the generation of telomere repeats copied from the telomerase-bound RNA template. Following elongation by telomerase, lagging strand synthesis by DNA polymerase completes formation of the double stranded telomeric structure [6].

In non-malignant human somatic cells, telomerase is not expressed or is expressed at low levels. Consequently, telomeres shorten by 50-200 bp with each cell division until the cells reach replicative senescence, at which point the cells lose the capacity to proliferate [7]. This limited capacity of cells to replicate is generally referred to as the Hayflick limit, and may provide cells with a counting mechanism, i.e., a mitotic clock, to count cell divisions and regulate cellular development [8-10]. Correspondingly, activation of telomerase in cells previously lacking telomerase activity, for example by expressing telomerase from a constitutive retroviral promoter or activation of endogenous polymerase, allows the cells to maintain proliferative capacity and leads to immortalization of the cell [11-21]. Unfortunately, to date, no clinically useful means of inducing a reduction in telomere shortening rate, a stabilization of telomere shortening, or an elongation of telomeres has been reported.

The transfection of human telomerase (hTERT) as a therapeutic modality is hindered by hurdles in uniformly transforming cells. An example of the drawbacks of current means of expanding, or elongating telomeres using approaches such as hTERT transfection can be seen in the hTERT transgenic mice which succumb to early onset neoplasia [22].

### BRIEF SUMMARY

The present disclosure is directed to methods and compositions for the modulation of telomere length. Certain aspects are directed to the use of fibroblasts for maintenance of telomere length, which may be useful in treatment or prevention of telomere-associated conditions such as aging, idiopathic pulmonary fibrosis, and cancer.

Disclosed herein, in some aspects, is a method for treating a telomere-associated condition in a subject comprising providing to the subject an effective amount of fibroblasts or fibroblast-derived products. In some embodiments, the method comprises providing to the subject an effective amount of fibroblasts. In some embodiments, the method further comprises, prior to the providing, inducing a pluripotent state in the fibroblasts. In some embodiments, the pluripotent state is a state which allows for differentiation of the fibroblasts into hematopoietic cells. In some embodiments, the hematopoietic cells are capable of multi-lineage reconstruction in an immune-deficient host. In some embodiments, the hematopoietic cells express c-kit, Sca-1, CD34, and/or CD33. In some embodiments, the hematopoietic cells do not express a lineage marker. In some embodiments, the hematopoietic cells do not express CD38. In some embodiments, the hematopoietic cells express c-kit and/or Sca-1, and the hematopoietic cells do not express a lineage marker. In some embodiments, the hematopoietic cells are capable of differentiating into granulocytes, monocytes, erythrocytes, thrombocytes, and/or lymphocytes. In some embodiments, the hematopoietic cells express TRA-1-60, SSEA-3, Sox2, Nanog, SSEA4, TRA-1-81, IGF1 receptor, connexin 43, E-cadherin, and/or Alkaline phosphatase. In some embodiments, inducing the pluripotent state comprises reprogramming the fibroblasts. In some embodiments, reprogramming the fibroblasts comprises transfection with Oct-4, Sox-2, Nanog, and/or Lin-28.

In some embodiments, the method comprises providing to the subject an effective amount of fibroblast-derived products. In some embodiments, the fibroblast-derived products comprise conditioned media derived from fibroblasts. In some embodiments, the fibroblast-derived products comprise microvesicles from fibroblasts. In some embodiments, the fibroblast-derived products comprise exosomes from fibroblasts. In some embodiments, the exosomes are between 30 nm and 150 nm in size. In some embodiments, the exosomes comprise a phospholipid, phosphatidyl serine, phosphatidyl inositol, phosphatidyl choline, sphingomyelin, ceramides, glycolipid, cerebroside, steroids, and/or cholesterol. In some embodiments, the exosomes comprise a lipid raft. In some embodiments, the exosomes comprise CD9, CD63, CD81, ANXA2, ENO1, HSP90AA1, EEF1A1, YWHAE, SDCBP, PDCD6IP, ALB, YWHAZ, EEF2, ACTG1, LDHA, HSP90AB1, ALDOA, MSN, ANXA5, PGK1, and/or CFL1. In some embodiments, the fibroblast-derived products comprise apoptotic vesicles from fibroblasts. In some embodiments, the fibroblast-derived products comprise nucleic acids from fibroblasts. In some embodiments, the nucleic acids are microRNAs.

In some embodiments, the fibroblasts or fibroblast-derived products are capable of modulating telomere length in cells of the subject. In some embodiments, the fibroblasts are capable of reducing the rate of shortening of the telomere length. In some embodiments, the fibroblasts are capable of preserving the telomere length. In some embodiments, the fibroblasts are capable of augmenting the telomere length. In some embodiments, the cells of the subject are hematopoietic cells. In some embodiments, the hematopoietic cells are leukocytes. In some embodiments, the leukocytes are peripheral blood mononuclear cells.

In some embodiments, the fibroblasts or fibroblast-derived products are provided to the subject intravenously, intralymphatically, intraperitoneally, intrathecally, intraventricularly, intra-arterially, or subcutaneously. In some embodiments, the fibroblasts express CD73, CD90, and/or CD105. In some embodiments, the fibroblasts express CD14, CD45, and/or CD34. In some embodiments, the fibroblasts are fibroblasts isolated from placenta, cord blood, peripheral blood, omentum, hair follicle, skin, bone marrow, adipose tissue, Wharton’s Jelly, umbilical cord tissue, skeletal muscle tissue, endometrial tissue, menstrual blood, and/or fallopian tube tissue. In some embodiments, the fibroblasts are isolated from umbilical cord tissue. In some embodiments, the fibroblasts express oxidized low density lipoprotein receptor 1, chemokine receptor ligand 3, and/or granulocyte chemotactic protein. In some embodiments, the fibroblasts do not express CD117, CD31, CD34, and/or CD45. In some embodiments, the fibroblasts express increased levels of interleukin 8 and/or reticulon 1 relative to a human mesenchymal stem cell. In some embodiments, the fibroblasts are capable of differentiating into skeletal muscle cells, vascular smooth muscle cells, pericytes, vascular endothelial cells, osteocytes, adipocytes, and/or chondrocytes.

In some embodiments, the fibroblasts express CD10, CD13, CD44, CD73, CD90, PDGFr-α, PD-L2, HLA-A, HLA-B, and/or HLA-C. In some embodiments, the fibroblasts do not express one or more of CD31, CD34, CD45, CD80, CD86, CD117, CD141, CD178, B7-H2, HLA-G, HLA-DR, HLA-DP, and/or HLA-DQ. In some embodiments, the fibroblasts secrete MCP-1, MIP1beta, IL-6, IL-8, GCP-2, HGF, KGF, FGF, HB-EGF, BDNF, TPO, RANTES, and/or TIMP1. In some embodiments, the fibroblasts express TRA1-60, TRA1-81, SSEA3, SSEA4, and/or NANOG.

In some embodiments, the method further comprises performing a cellular graft in the subject, where the fibroblasts or fibroblast-derived products increase the quality of the cellular graft. In some embodiments, the cellular graft is an islet transplant, a hepatocyte transplant, or a hematopoietic stem cell transplant. In some embodiments, the method further comprises providing one or more lithium-containing compound,s or a pharmaceutically acceptable salt thereof, to the subject. In some embodiments, the lithium-containing compound is lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, and/or lithium succinate.

In some embodiments, the telomere-associated condition is dyskeratosis congenita, cancer, cellular senescence, idiopathic pulmonary fibrosis, Hoyeraal-Hreiderasson syndrome, Hutchinson-Gilford progeria, aplastic anemia, or an age-related disease. In some embodiments, the telomere-associated condition is aging, idiopathic pulmonary fibrosis, or cancer.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, the term “capable of” refers to having the activity of, in at least some aspects.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “telomere-associated condition,” as used herein, refers to any condition or disorder associated with abnormal telomeres in cells of a subject. Certain types telomere-associated conditions are those associated with abnormally short telomeres or telomeres which shorten at an abnormally fast rate in cells of an individual. Such conditions may arise due to decreased telomerase activity and include, for example, idiopathic pulmonary fibrosis, dyskeratosis congenita, Hoyeraal-Hreiderasson syndrome, Hutchinson-Gilford progeria, aplastic anemia, aging, and age-related diseases. The methods and compositions of the present disclosure may be used to lengthen telomeres or to slow the rate of telomere shortening in these cases. Other types of telomere-associated conditions are those associated with abnormally long telomeres or telomeres which shorten at an abnormally slow rate in cells of an individual. Such conditions may arise due to increased telomerase activity and include, for example, cancer. The methods and compositions of the present disclosure may be used to shorten telomeres or to increase the rate of telomere shortening in these cases.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles derived from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

### II. Modulation of Telomere Length

Aspects of the disclosure are based on the unexpected finding that administration of fibroblasts, or fibroblast-derived products, can be used to modulate telomere length in cells of a subject. In some embodiments, fibroblasts of the present disclosure are used to increase telomere length in a subject. In particular, some embodiments are directed to the use of fibroblasts or fibroblast-derived products to inhibit the rate of telomere shortening, stabilize telomere length, and/or elongate telomere length in a subject. In one embodiment, disclosed are methods for enhancing telomere length in cells of an individual comprising providing fibroblast cells to the individual. Enhancement of telomere length may be beneficial in situations where an inhibition of cellular senescence is desired, for example in aging and age-related disorders.

Fibroblasts or fibroblast-derived products may be used to modulate (e.g., enhance) telomere length in one or more types of cells in a subject. In some embodiments, fibroblasts or fibroblast-derived products are used to modulate telomere length in one or more of endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner’s gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman’s gland cells, Brunner’s gland cells, seminal vesicle cells, prostate gland cells, bulbourethral gland cells, Bartholin’s gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cells, blood vessel and lymphatic vascular endothelial fenestrated cells, blood vessel and lymphatic vascular endothelial continuous cells, synovial cells, serosal cells, squamous cells, columnar cells, dark cells, vestibular membrane cells, stria vascularis basal cells, stria vascularis marginal cells, cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley’s layer, hair root sheath cells of Henle’s layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells, hepatic stellate cells, pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and interstitial kidney cells. In some embodiments, the disclosed methods comprise use of fibroblasts or fibroblast-derived products to modulate telomere length in endothelial progenitor cells.

In some embodiments, fibroblasts or fibroblast-derived products are used to modify telomere length in malignant cells, including cancer cells. For example, fibroblasts or fibroblast-derived products may be used to shorten telomere length in cancer cells, thus stimulating cancer cell death.

### III. Methods and Compositions for Disease Treatment or Prevention

Aspects of the present disclosure are directed to methods and compositions for treatment or prevention of telomere-associated conditions. Telomeres are known to be involved in a number of diseases and medical conditions. The telomere-associated conditions treated or prevented using methods and compositions of the disclosure include, for example, dyskeratosis congenita, cancer, cellular ageing (cellular senescence), idiopathic pulmonary fibrosis, Hoyeraal-Hreiderasson syndrome, Hutchinson-Gilford progeria, aplastic anemia, and age-related diseases. Age-related diseases include, for example, osteoporosis, type II diabetes, atherosclerosis and cardiovascular disease.

In some embodiments, fibroblast cell administration is used to increase telomere length in conditions associated with telomere shortening. For example, studies have shown correlations between shortening of leukocyte telomeres and cardiac conditions. In one study, Haver et al. assessed leucocyte telomere length in 3275 patients with chronic ischaemic systolic heart failure participating in the COntrolled ROsuvastatin multiNAtional Trial in Heart Failure (CORONA) study. The primary composite endpoint was cardiovascular death, non-fatal myocardial infarction, and non-fatal stroke, which occurred in 575 patients during follow-up. They observed a significant association of leucocyte telomere lengths with the primary endpoint (hazard ratio 1.10; 95% confidence interval 1.01-1.20; P=0.03) [32]. One therapeutic cell type which may be targeted by the present disclosure is the endothelial progenitor cell (EPC), whose activity has been demonstrated to correlate with superior cardiovascular health [33-35]. Indeed telomere length has been shown to correlate with EPC activity [36-39]. Numerous factors contribute to telomere shortening in addition to aging, including DNA damage, inflammation, and oxidative stress. Both cardiovascular risk factors and common cardiovascular diseases, such as atherosclerosis, heart failure, and hypertension, are associated with short leukocyte telomeres [40].

In some embodiments, disclosed herein are methods for treating telomere-associated conditions comprising providing to a subject an effective amount of fibroblasts or fibroblast-associated products. In some embodiments, disclosed are methods comprising providing an effective amount of fibroblasts. In some embodiments, a pluripotent state is induced in the fibroblasts prior to providing the fibroblasts to the subject. A pluripotent state may be a state that allows differentiation of the fibroblasts into hematopoietic cells. In some embodiments, fibroblasts of the present disclosure differentiate into hematopoietic cells before or after providing the cells to the subject. Hematopoietic cells of the disclosure may be capable of capable of multi-lineage reconstruction, such as in an immune-deficient host. In some embodiments, hematopoietic cells express c-kit, Sca-1, CD34, or CD33. In some embodiments, hematopoietic cells do not express a lineage marker. In some embodiments, hematopoietic cells do not express CD38. In some embodiments, hematopoietic cells express c-kit and Sca-1, and wherein the hematopoietic cells do not express a lineage marker. In some embodiments, hematopoietic cells are capable of differentiating into granulocytes, monocytes, erythrocytes, thrombocytes, or lymphocytes. In some embodiments, the hematopoietic cells express TRA-1-60, SSEA-3, Sox2, Nanog, SSEA4, TRA-1-81, IGF1 receptor, connexin 43, E-cadherin, or Alkaline phosphatase. In some embodiments, a pluripotent state is induced in the fibroblasts by reprogramming the fibroblasts. Reprogramming fibroblasts may comprise transfection with Oct-4, Sox-2, Nanog, or Lin-28.

In some embodiments, disclosed are methods comprising providing an effective amount of fibroblast-derived products. Examples of fibroblast-derived products used in the methods and compositions of the present disclosure include conditioned media, microvesicles, exosomes, apoptotic vesicles, and nucleic acids. Exosomes may be between 30 nm and 150 nm in size. Exosomes may comprise a phospholipid, phosphatidyl serine, phosphatidyl inositol, phosphatidyl choline, sphingomyelin, ceramides, glycolipid, cerebroside, steroids, or cholesterol. Exosomes may comprise a lipid raft. In some embodiments, exosomes from fibroblasts comprise CD9, CD63, CD81, ANXA2, ENO1, HSP90AA1, EEF1A1, YWHAE, SDCBP, PDCD6IP, ALB, YWHAZ, EEF2, ACTG1, LDHA, HSP90AB1, ALDOA, MSN, ANXA5, PGK1, or CFL1. Nucleic acids from fibroblasts include, for example, microRNAs (miRNAs).

In some embodiments, fibroblasts are provided to modulate telomere length in cells of an individual. In some embodiments, fibroblasts preserve the telomere length in the cells of the individual. In some embodiments, fibroblasts increase the telomere length in the cells of the individual. In some embodiments, fibroblasts are provided to modulate telomere length in hematopoietic cells in a subject. In some embodiments, the hematopoietic cells are leukocytes (e.g., peripheral blood mononuclear cells).

In some embodiments, the fibroblasts provided for modulation of telomere length express CD73, CD90, or CD105. In some embodiments, the fibroblasts express CD14, CD45, or CD34. Fibroblasts used in the disclosed methods may be derived from any source. In some embodiments, fibroblasts are derived from umbilical cord tissue. In some embodiments, the fibroblasts express oxidized low density lipoprotein receptor 1, chemokine receptor ligand 3, or granulocyte chemotactic protein. In some embodiments, the fibroblasts do not express CD117, CD31, CD34, or CD45. In some embodiments, the fibroblasts express increased levels of interleukin 8 and reticulon 1 relative to a human mesenchymal stem cell.

In some embodiments, the fibroblasts are capable of differentiating into skeletal muscle cells, vascular smooth muscle cells, pericytes, vascular endothelial cells, osteocytes, adipocytes, or chondrocytes. In some embodiments, the fibroblasts express CD10, CD13, CD44, CD73, CD90, PDGFr-α, PD-L2, HLA-A, HLA-B, or HLA-C. In some embodiments, the fibroblasts do not express one or more of CD31, CD34, CD45, CD80, CD86, CD117, CD141, CD178, B7-H2, HLA-G, HLA-DR, HLA-DP, or HLA-DQ. In some embodiments, the fibroblasts secrete MCP-1, MIP1beta, IL-6, IL-8, GCP-2, HGF, KGF, FGF, HB-EGF, BDNF, TPO, RANTES, or TIMP1. In some embodiments, the fibroblasts express TRA1-60, TRA1-81, SSEA3, SSEA4, or NANOG.

In some embodiments, methods of the present disclosure further comprise, in addition to providing an effective amount of fibroblasts or fibroblast-derived products, performing a cellular graft on a subject. In some embodiments, the fibroblasts or fibroblast-derived products increase the quality of the cellular graft. The cellular graft may be, for example, cellular graft is an islet transplant, a hepatocyte transplant, or a hematopoietic stem cell transplant. In some embodiments, methods of the present disclosure further comprise, in addition to providing an effective amount of fibroblasts or fibroblast-derived products, providing a lithium-containing compound, or a pharmaceutically acceptable salt thereof, to a subject. Examples of lithium-containing compounds useful in the present disclosure include lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, or lithium succinate.

In some embodiments, fibroblasts (e.g., allogenic fibroblasts) are provided in a non-manipulated manner, but selected from a source characterized by immune modulatory activity (e.g., placental fibroblasts). Alternatively or in addition, in some embodiments, fibroblasts are cultured under conditions capable of inducing retrodifferentiation so as to endow an immature phenotype, the immature phenotype correlating with enhanced ability to restore telomere length. For example, fibroblasts may be cultured in the presence of a histone deacetylase inhibitor such as valproic acid. Conditions of growing cells in HDAC inhibitors to induce dedifferentiation are described previously and incorporated herein by reference [41, 42]. Other means of inducing dedifferentiation in addition to HDAC inhibitors may also be utilized in the context of the current invention including 8-Br-cAMP [43], M-CSF treatment [44], exposure to reveresine [45], and exposure to stem cell extracts [46]. Quantification of fibroblast dedifferentiation can be performed by assessment of extracellular markers, as well as intracellular markers such as SOX-2, NANOG, and OCT-4.

In some embodiments, prior to providing fibroblasts to a subject, the fibroblasts are cultured with an agent capable of protecting the cells from in vitro aging as described further herein. In some embodiments, prior to providing fibroblasts to a subject, the fibroblasts are cultured with a proteolysis inhibitor (e.g., a protease inhibitor, a proteasome inhibitor, a lysosome inhibitor) as described further herein. In some embodiments, prior to providing fibroblasts to a subject, the fibroblasts are cultured with an inhibitor of mRNA degradation, as described further herein.

Guidance is given to one of skill in the art in the practice of the invention by publications in the field, for example, Townsley et al treated 27 patients suffering from accelerated telomere shortening with the synthetic sex hormone danazol orally at a dose of 800 mg per day for a total of 24 months. The goal of treatment was the attenuation of accelerated telomere attrition, and the primary efficacy end point was a 20% reduction in the annual rate of telomere attrition measured at 24 months. The occurrence of toxic effects of treatment was the primary safety end point. Hematologic response to treatment at various time points was the secondary efficacy end point. Telomere attrition as part of the disease pathology was reduced in all 12 patients who could be evaluated for the primary end point; in the intention-to-treat analysis, 12 of 27 patients (44%; 95% confidence interval [CI], 26 to 64) met the primary efficacy end point. Unexpectedly, almost all the patients (11 of 12, 92%) had a gain in telomere length at 24 months as compared with baseline (mean increase, 386 bp [95% CI, 178 to 593]); in exploratory analyses, similar increases were observed at 6 months (16 of 21 patients; mean increase, 175 bp [95% CI, 79 to 271]) and 12 months (16 of 18 patients; mean increase, 360 bp [95% CI, 209 to 512]). Hematologic responses occurred in 19 of 24 patients (79%) who could be evaluated at 3 months and in 10 of 12 patients (83%) who could be evaluated at 24 months. Known adverse effects of danazol—elevated liver-enzyme levels and muscle cramps—of grade 2 or less occurred in 41% and 33% of the patients, respectively [47].

Assessment of telomere length may be performed using means previously described and incorporated by reference [48-51]

### IV. Fibroblasts and Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells. In one example, fibroblasts are isolated and provided to a subject together with one or more endothelial progenitor cells. In some embodiments, disclosed herein are fibroblasts capable of modulating telomere length. In some embodiments, fibroblasts of the present disclosure are adherent to plastic, express CD73, CD90, and CD105 antigens, while being CD14, CD34, CD45, and HLA-DR negative, and possess the ability to differentiate to osteogenic, chondrogenic, and adipogenic lineage cells.

Compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, fibroblasts of the present disclosure express telomerase, Nanog, Sox2, (β-III-Tubulin, NF-M, MAP2, APP, GLUT, NCAM, NeuroD, Nurrl, GFAP, NG2, Olig1, Alkaline Phosphatase, Vimentin, Osteonectin, Osteoprotegrin, Osterix, Adipsin, Erythropoietin, SM22-α, HGF, c-MET, .alpha.-1-Antriptrypsin, Ceruloplasmin, AFP, PEPCK 1, BDNF, NT-⅘, TrkA, BMP2, BMP4, FGF2, FGF4, PDGF, PGF, TGF.alpha., TGF.beta., and/or VEGF.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells of the invention herein comprises Dulbecco’s Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, Hyclone™, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick’s limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be obtained from a source selected from the group consisting of: dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and a combination thereof. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-y, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote angiogenesis. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of treatment or prevention of telomere-associated conditions.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., hematopoietic cells) prior to introduction into the individual.

As disclosed herein, fibroblasts may secret one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with an agent capable of protecting the cells from in vitro aging. In some embodiments, the agent is reversin, cord blood serum, lithium, a GSK-3 inhibitor, resveratrol, pterostilbene, selenium, a selenium-containing compound, EGCG ((-)-epigallocatechin-3-gallate), valproic acid, or a salt of valproic acid (e.g., sodium valproate). In some embodiments, fibroblasts are cultured with reversin at a concentration of between 0.5 µM and 10 µM, for example about 1 µM. In some embodiments, fibroblasts are cultured with resveratrol at 10 µM and 100 µM, for example about 50 µM. In some embodiments, fibroblasts are cultured with selenium or a selenium containing compound at a concentration of between 0.05 µM and 0.5 µM, for example about 0.1 µM. In some embodiments, fibroblasts are cultured with cord blood serum at a concentration of between .1% and 20% volume to the volume of tissue culture media. In some embodiments, fibroblasts are cultured with EGCG at a concentration of between 0.001 µM and 0.1 µM, for example about 0.01 µM. In some embodiments, fibroblasts are cultured with valproic acid or sodium valproate at a concentration of between 1 µM and 10 µM, for example about 5 µM.

In some embodiments, fibroblasts of the present disclosure are cultured with a proteolysis inhibitor. A proteolysis inhibitor may be useful in, for example, improving efficacy of fibroblast reprogramming. A proteolysis inhibitor describes an agent or compound capable of inhibiting proteolysis in a cell, such as, for example, a protease inhibitor, a proteasome inhibitor or a lysosome inhibitor. In some embodiments, fibroblasts are cultured with a protease inhibitor. In some embodiments, the protease inhibitor is aprotinin, G-64, or leupeptine. In some embodiments, fibroblasts are cultured with a proteasome inhibitor. In some embodiments, the proteasome inhibitor is MG132, TMC-95A, TS-341, or MG262. In some embodiments, fibroblasts are cultured with a lysosome inhibitor. In some embodiments, the lysosome inhibitor is ammonium chloride.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Aspects of the present disclosure comprise generating conditioned media from fibroblasts. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500 xg). Conditioned media may be filtered through a membrane. The membrane may be a >1000 kDa membrane. Conditioned media may be subject to liquid chromatography such as HPLC. Conditioned media may be separated by size exclusion.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of for example treating or preventing a telomere-associated condition. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

### V. Fibroblast Dedifferentiation and Differentiation

In some embodiments, fibroblasts of the present disclosure are dedifferentiated prior to administration to a subject and/or use in modulating telomere length. In some embodiments, fibroblasts are transfected or transduced with an Oct-4 reporter gene. In some embodiments, the Oct-4 reporter gene is introduced by lentiviral transduction. The term “Oct-4-reporter” as used herein refers to DNA sequences that are bound by Oct-4 upstream of a reporter that allow or enhance transcription of the downstream sequences of the reporter. Example Oct-4 reporters are described in, for example, Hotta et al., Nat Methods, 6 (5), 370-6, 2009 and Okumura-Nakanishi et al., J Biol Chem, 280 (7), 5307-17, 2005, incorporated herein by reference in their entireties. In some embodiments, disclosed are methods for generating a fibroblast-derived induced pluripotent stem (iPS) cell. In some embodiments, generating a fibroblast-derived iPS cell comprises providing Oct-4, Sox-2, Nanog, and Lin-28 to fibroblasts and culturing the cells under conditions sufficient to transform the fibroblasts into iPS cells. In some embodiments, Oct-4, Sox-2, Nanog, and Lin-28 are provided to the fibroblasts by viral transduction (e.g., lentiviral transduction).

The term “stem cell” as used herein refers to a cell that has the ability for self-renewal. In one embodiment, the stem cell is a pluripotent stem cell. The term “pluripotent” as used herein refers to an undifferentiated cell that maintains the ability to allow differentiation into various cell types. The term “induced pluripotent stem cell” refers to a pluripotent stem cell that has been artificially derived from a non-pluripotent stem cell. The term “Sox-2” as used herein refers to the gene product of the Sox-2 gene and includes Sox-2 from any species or source and includes variants, analogs and fragments or portion of Sox-2 that retain activity. The Sox-2 protein may have any of the known published sequences for Sox-2, which can be obtained from public sources such as GenBank. An example of such a sequence includes, but is not limited to, NM_003106. The term “Nanog” as used herein refers to the gene product of the Nanog gene and includes Nanog from any species or source and includes variants, analogs and fragments or portion of Nanog that retain activity. The Nanog protein may have any of the known published sequences for Nanog, which can be obtained from public sources such as GenBank. An example of such a sequence includes, but is not limited to, NM_024865. The term “Lin-28” as used herein refers to the gene product of the Lin-28 gene and includes Lin-28 from any species or source and includes variants, analogs and fragments or portions of Lin-28 that retain activity. The Lin-28 protein may have any of the known published sequences for Lin 28, which can be obtained from public sources such as GenBank. An example of such a sequence includes, but is not limited to, BC028566.2. Lin-28 is also called CSDD1 or ZCCHC1 or Lin28A.

In some embodiments, generation of cells for use in the methods of the present disclosure comprises dedifferentiation using mRNA transfer. In some embodiments, RNA or mRNA is extracted to achieve pluripotency in the ‘target’ cells Examples of recipient or target cells into which RNA or mRNA can be introduced to achieve pluripotency or transdifferentiation in the ‘target’ cells include, by way of example, primary fibroblasts. Various sources of fibroblasts may be used, depending on tissue and age. Examples of somatic cells which may be used as the donor cell for transdifferentiation include any cell type that is desired for cell therapies including, by way of example, hepatocytes, lymphocytes, beta cells, neural cells, cardiac cells, and lung cells. In some embodiments, fibroblasts are dedifferentiated using total RNA or mRNA. The mRNA or total RNA used to effect dedifferentiation may be isolated from cells that are either pluripotent or which are capable of turning into pluripotent cells (oocyte). Examples thereof include, by way of example, Ntera (e.g., Ntera-2) cells, human or other ES cells, primordial germ cells, and blastocysts. Alternatively the RNA used to effect dedifferentiation may comprise mRNA encoding specific transcription factors. The total RNA or mRNA may be delivered into target cells by various methods including e.g., electroporation, liposomes, and mRNA injection. Target cells into which RNA’s are introduced and which are to be dedifferentiated may be cultured in a medium containing one or more constituents that facilitates transformation of cell phenotype. These constituents include, by way of example, epigenetic modifiers (e.g., DNA demethylating agents, HDAC inhibitors, histone modifiers), helper cells capable of promoting growth of pluripotent cells, growth factors, hormones, and bioactive molecules. Examples of DNA methylating agents include 5-azacytidine (5-aza), MNNG, 5-aza, N-methl-N′-nitro-N-nitrosoguanidine, temozolomide, and procarbazine. Examples of methylation inhibiting drugs agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, and other inhibitors of enzymes involved in the methylation of DNA. Examples of histone deacetylase (“HDAC”) inhibitors include hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Examples of histone modifiers include PARP, the human enhancer of zeste, valproic acid, and trichostatine. In some embodiments, the components included in media for facilitating RNA transformation and dedifferentiation of the RNA-containing target cells into pluripotent cells include trichostatine, valproic acid, zebularine and 5-aza. Target cells into which RNA is introduced are cultured for a sufficient time in media that promotes RNA transformation until dedifferentiated (pluripotent) cells are obtained. One embodiment of the disclosure teaches introduction of total RNA or mRNA from one cell type such as a pluripotent or somatic cell into a desired human somatic cell such as a fibroblast in order to dedifferentiate or transdifferentiate such cell into a pluripotent cell or a different somatic cell corresponding to the lineage of the cell from which the donor total RNA is derived. This may be sufficient to effect cell dedifferentiation or transdifferentiation. In some instances this methodology may be combined with other methods and treatments involved in the epigenetic status of the recipient or target cell such as the exposure to DNA and histone demethylating agents, histone deacetylase inhibitors, and/or histone modifiers. By using epigenetic modifications, the subject methods can dedifferentiate or transdifferentiate cells. Aspects of this disclosure are aimed to solve the problem of immuno-rejection which is evident when incompatible cells and/or tissues are used for transplantation.

### VI. Administration of Therapeutic Compositions

The therapy provided herein may comprise administration of a therapeutic agents alone or in combination. Therapies may be administered in any suitable manner known in the art. For example, a first and second treatment may be administered sequentially (at different times) or concurrently (at the same time, also “simultaneously”). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual’s clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 µM to 150 µM. In another embodiment, the effective dose provides a blood level of about 4 µM to 100 µM.; or about 1 µM to 100 µM; or about 1 µM to 50 µM; or about 1 µM to 40 µM; or about 1 µM to 30 µM; or about 1 µM to 20 µM; or about 1 µM to 10 µM; or about 10 µM to 150 µM; or about 10 µM to 100 µM; or about 10 µM to 50 µM; or about 25 µM to 150 µM; or about 25 µM to 100 µM; or about 25 µM to 50 µM ; or about 50 µM to 150 µM; or about 50 µM to 100 µM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 µM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of µg/kg or mg/kg of body weight can be converted and expressed in comparable concentration units of µg/ml or mM (blood levels), such as 4 µM to 100 µM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### VII. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Melicher, D., E.I. Buzas, and A. Falus, Genetic and epigenetic trends in telomere research: a novel way in immunoepigenetics. Cell Mol Life Sci, 2015. 72(21): p. 4095-109.
2. Dubrana, K., S. Perrod, and S.M. Gasser, Turning telomeres off and on. Curr Opin Cell Biol, 2001. 13(3): p. 281-9.
3. Counter, C.M., The roles of telomeres and telomerase in cell life span. Mutat Res, 1996. 366(1): p. 45-63.
4. Fajkus, J., E. Sykorova, and A.R. Leitch, Telomeres in evolution and evolution of telomeres. Chromosome Res, 2005. 13(5): p. 469-79.
5. Cervantes, R.B. and V. Lundblad, Mechanisms of chromosome-end protection. Curr Opin Cell Biol, 2002. 14(3): p. 351-6.
6. Moon, I.K. and M.B. Jarstfer, The human telomere and its relationship to human disease, therapy, and tissue engineering. Front Biosci, 2007. 12: p. 4595-620.
7. Baird, D.M., Mechanisms of telomeric instability. Cytogenet Genome Res, 2008. 122(3-4): p. 308-14.
8. Johnson, F.B., R.A. Marciniak, and L. Guarente, Telomeres, the nucleolus and aging. Curr Opin Cell Biol, 1998. 10(3): p. 332-8.
9. Toussaint, O., et al., From the Hayflick mosaic to the mosaics of ageing. Role of stress-induced premature senescence in human ageing. Int J Biochem Cell Biol, 2002. 34(11): p. 1415-29.
10. Awaya, N., et al., Telomere shortening in hematopoietic stem cell transplantation: a potential mechanism for late graft failure? Biol Blood Marrow Transplant, 2002. 8(11): p. 597-600.
11. Vidale, P., et al., The catalytic and the RNA subunits of human telomerase are required to immortalize equid primary fibroblasts. Chromosoma, 2012. 121(5): p. 475-88.
12. Qiao, B., et al., Epithelial-mesenchymal transition and mesenchymal-epithelial transition are essential for the acquisition of stem cell properties in hTERT-immortalised oral epithelial cells. Biol Cell, 2012. 104(8): p. 476-89.
13. Mazzucchelli, G.D., et al., Proteome alteration induced by hTERT transfection of human fibroblast cells. Proteome Sci, 2008. 6: p. 12.
14. Jesnowski, R., et al., Immortalization of pancreatic stellate cells as an in vitro model of pancreatic fibrosis: deactivation is induced by matrigel and N-acetylcysteine. Lab Invest, 2005. 85(10): p. 1276-91.
15. Pirzio, L.M., et al., Human fibroblasts expressing hTERT show remarkable karyotype stability even after exposure to ionizing radiation. Cytogenet Genome Res, 2004. 104(1-4): p. 87-94.
16. Veitonmaki, N., et al., Immortalization of bovine capillary endothelial cells by hTERT alone involves inactivation of endogenous p16INK4A/pRb. FASEB J, 2003. 17(6): p. 764-6.
17. Schnabl, B., et al., Immortal activated human hepatic stellate cells generated by ectopic telomerase expression. Lab Invest, 2002. 82(3): p. 323-33.
18. Cerone, M.A., J.A. Londono-Vallejo, and S. Bacchetti, Telomere maintenance by telomerase and by recombination can coexist in human cells. Hum Mol Genet, 2001. 10(18): p. 1945-52.
19. O’Hare, M.J., et al., Conditional immortalization of freshly isolated human mammary fibroblasts and endothelial cells. Proc Natl Acad Sci USA, 2001. 98(2): p. 646-51.
20. Wen, J., Y.S. Cong, and S. Bacchetti, Reconstitution of wild-type or mutant telomerase activity in telomerase-negative immortal human cells. Hum Mol Genet, 1998. 7(7): p. 1137-41.
21. Counter, C.M., et al., Telomerase activity is restored in human cells by ectopic expression of hTERT (hEST2), the catalytic subunit of telomerase. Oncogene, 1998. 16(9): p. 1217-22.
22. Horikawa, I., et al., Differential cis-regulation of human versus mouse TERT gene expression in vivo: identification of a human-specific repressive element. Proc Natl Acad Sci U S A, 2005. 102(51): p. 18437-42.
23. Cronkhite, J.T., et al., Telomere shortening in familial and sporadic pulmonary fibrosis. Am J Respir Crit Care Med, 2008. 178(7): p. 729-37.
24. Alder, J.K., et al., Short telomeres are a risk factor for idiopathic pulmonary fibrosis. Proc Natl Acad Sci USA, 2008. 105(35): p. 13051-6.
25. Diaz de Leon, A., et al., Telomere lengths, pulmonary fibrosis and telomerase (TERT) mutations. PLoS One, 2010. 5(5): p. e10680.
26. Alder, J.K., et al., Telomere length is a determinant of emphysema susceptibility. Am J Respir Crit Care Med, 2011. 184(8): p. 904-12.
27. Tsang, A.R., et al., hTERT mutations associated with idiopathic pulmonary fibrosis affect telomerase activity, telomere length, and cell growth by distinct mechanisms. Aging Cell, 2012. 11(3): p. 482-90.
28. Faner, R., et al., Abnormal lung aging in chronic obstructive pulmonary disease and idiopathic pulmonary fibrosis. Am J Respir Crit Care Med, 2012. 186(4): p. 306-13.
29. Fernandez, B.A., et al., A Newfoundland cohort of familial and sporadic idiopathic pulmonary fibrosis patients: clinical and genetic features. Respir Res, 2012. 13: p. 64.
30. Armanios, M., Telomeres and age-related disease: how telomere biology informs clinical paradigms. J Clin Invest, 2013. 123(3): p. 996-1002.
31. Codd, V., et al., Identification of seven loci affecting mean telomere length and their association with disease. Nat Genet, 2013. 45(4): p. 422-7, 427e1-2.
32. Haver, V.G., et al., Telomere length and outcomes in ischaemic heart failure: data from the COntrolled ROsuvastatin multiNAtional Trial in Heart Failure (CORONA). Eur J Heart Fail, 2015. 17(3): p. 313-9.
33. Alba, A.C., et al., Changes in circulating progenitor cells are associated with outcome in heart failure patients: a longitudinal study. Can J Cardiol, 2013. 29(12): p. 1657-64.
34. Chu, K., et al., Circulating endothelial progenitor cells as a new marker of endothelial dysfunction or repair in acute stroke. Stroke, 2008. 39(5): p. 1441-7.
35. Sobrino, T., et al., The increase of circulating endothelial progenitor cells after acute ischemic stroke is associated with good outcome. Stroke, 2007. 38(10): p. 2759-64.
36. Vemparala, K., et al., Early accelerated senescence of circulating endothelial progenitor cells in premature coronary artery disease patients in a developing country - a case control study. BMC Cardiovasc Disord, 2013. 13: p. 104.
37. Olivieri, F., et al., Cellular senescence in cardiovascular diseases: potential age-related mechanisms and implications for treatment. Curr Pharm Des, 2013. 19(9): p. 1710-9.
38. Satoh, M., et al., Association between oxidative DNA damage and telomere shortening in circulating endothelial progenitor cells obtained from metabolic syndrome patients with coronary artery disease. Atherosclerosis, 2008. 198(2): p. 347-53.
39. Kushner, E.J., et al., Aging and endothelial progenitor cell telomere length in healthy men. Clin Chem Lab Med, 2009. 47(1): p. 47-50.
40. Fyhrquist, F., O. Saijonmaa, and T. Strandberg, The roles of senescence and telomere shortening in cardiovascular disease. Nat Rev Cardiol, 2013. 10(5): p. 274-83.
41. Moon, J.H., et al., Induction of neural stem cell-like cells (NSCLCs)from mouse astrocytes by Bmil. Biochem Biophys Res Commun, 2008. 371(2): p. 267-72.
42. Huang, Y., et al., Histone deacetylase inhibitor significantly improved the cloning efficiency of porcine somatic cell nuclear transfer embryos. Cell Reprogram, 2011. 13(6): p. 513-20.
43. Wang, Y. and J. Adjaye, A cyclic AMP analog, 8-Br-cAMP, enhances the induction of pluripotency in human fibroblast cells. Stem Cell Rev, 2011. 7(2): p. 331-41.
44. Li, Y., R.B. Jalili, and A. Ghahary, Accelerating skin wound healing by M-CSF through generating SSEA-1 and -3 stem cells in the injured sites. Sci Rep, 2016. 6: p. 28979.
45. Li, X., et al., Reversine Increases the Plasticity of Long-Term Cryopreserved Fibroblasts to Multipotent Progenitor Cells through Activation of Oct4. Int J Biol Sci, 2016. 12(1): p. 53-62.
46. Xiong, X.R., et al., Cellular extract facilitates nuclear reprogramming by altering DNA methylation and pluripotency gene expression. Cell Reprogram, 2014. 16(3): p. 215-22.
47. Townsley, D.M., et al., Danazol Treatment for Telomere Diseases. N Engl J Med, 2016. 374(20): p. 1922-31.
48. Baljevic, M., et al., Telomere Length Recovery: A Strong Predictor of Overall Survival in Acute Promyelocytic Leukemia. Acta Haematol, 2016. 136(4): p. 210-218.
49. Barden, A., et al., n-3 Fatty Acid Supplementation and Leukocyte Telomere Length in Patients with Chronic Kidney Disease. Nutrients, 2016. 8(3): p. 175.
50. Harris, S.E., et al., Longitudinal telomere length shortening and cognitive and physical decline in later life: The Lothian Birth Cohorts 1936 and 1921. Mech Ageing Dev, 2016. 154: p. 43-8.
51. Gu, Y., et al., Telomere length, genetic variants and risk of squamous cell carcinoma of the head and neck in Southeast Chinese. Sci Rep, 2016. 6: p. 20675.

1. A method for treating a telomere-associated condition in a subject comprising providing to the subject an effective amount of fibroblasts, conditioned media from fibroblast culture, microvesicles obtained from fibroblasts, exosomes obtained from fibroblasts, apoptotic vesicles derived from fibroblasts, nucleic acids obtained from fibroblasts, proteins obtained from fibroblasts, lipids obtained from fibroblasts, and/or fibroblast-derived products.

2. The method of claim 1, wherein the method comprises providing to the subject an effective amount of fibroblasts.

3. The method of claim 1, wherein the method further comprises, prior to the providing, inducing a pluripotent state in the fibroblasts.

4. The method of claim 3, wherein the pluripotent state is a state that allows for differentiation of the fibroblasts into hematopoietic cells.

5. The method of claim 4, wherein the hematopoietic cells are capable of multilineage reconstruction in an immune-deficient host.

6. The method of claim 4, wherein:
(a) the hematopoietic cells express c-kit, Sca-1, CD34, and/or CD33;
(b) the hematopoietic cells do not express a lineage marker;
(c) the hematopoietic cells do not express CD38;
(d) the hematopoietic cells express c-kit and Sca- 1, and wherein the hematopoietic cells do not express a lineage marker;
(e) the hematopoietic cells are capable of differentiating into granulocytes, monocytes, erythrocytes, thrombocytes, and/or lymphocytes; and/or
(f) the hematopoietic cells express TRA-1-60, SSEA-3, Sox2, Nanog, SSEA4, TRA-1-81, IGF1 receptor, connexin 43, E-cadherin, and/or Alkaline phosphatase.

7. (canceled)

8. (canceled)

9. (canceled)

10. (canceled)

11. (canceled)

12. The method of claim 4, wherein inducing the pluripotent state comprises reprogramming the fibroblasts.

13. The method of claim 12, wherein reprogramming the fibroblasts comprises transfection of the fibroblasts with Oct-4, Sox-2, Nanog, and/or Lin-28.

14. The method of claim 1, wherein the method comprises providing to the subject an effective amount of fibroblast-derived products.

15. The method of claim 14, wherein the fibroblast-derived products comprise conditioned media derived from fibroblasts, microvesicles from fibroblasts, and/or exosomes from fibroblasts.

16. (canceled)

17. (canceled)

18. The method of claim 15, wherein the exosomes are between 30 nm and 150 nm in size.

19. The method of claim 15, wherein the exosomes comprise;
(a) a phospholipid, phosphatidyl serine, phosphatidyl inositol, phosphatidyl choline, sphingomyelin, ceramides, glycolipid, cerebroside, steroids, or cholesterol;
(b) a lipid raft and/or
(c) CD9, CD63, CD81, ANXA2, ENOl, HSP90AA1, EEF1A1, YWHAE, SDCBP, PDCD6IP, ALB, YWHAZ, EEF2, ACTG1, LDHA, HSP90AB1, ALDOA, MSN, ANXA5, PGK1, and/or CFL1.

20. (canceled)

21. (canceled)

22. The method of claim 14, wherein the fibroblast-derived products comprise apoptotic vesicles from fibroblasts and/or nucleic acids from fibroblasts.

23. (canceled)

24. The method of claim 22, wherein the nucleic acids are microRNAs.

25. The method of claim 1, wherein the fibroblasts or fibroblast-derived products are capable of modulating telomere length in cells of the subj ect.

26. The method of claim 25, wherein the fibroblasts are capable of reducing the rate of shortening of the telomere length, preserving the telomere length, and/or augmenting the telomere length.

27. (canceled)

28. (canceled)

29. The method of claim 25, wherein the cells of the subject are hematopoietic cells.

30. The method of claim 29, wherein the hematopoietic cells are leukocytes.

31. The method of claim 30, wherein the leukocytes are peripheral blood mononuclear cells.

32. The method of claim 1, wherein the fibroblasts or fibroblast-derived products are provided to the subject intravenously, intralymphatically, intraperitoneally, intrathecally, intraventricularly, intra-arterially, or subcutaneously.

33. The method of claim 1, wherein the fibroblasts express CD73, CD90, CD105, CD14, CD45, and/or CD34.

34. (canceled)

35. The method of claim 1, wherein the fibroblasts are fibroblasts isolated from placenta, cord blood, peripheral blood, omentum, hair follicle, skin, bone marrow, adipose tissue, Wharton’s Jelly, umbilical cord tissue, skeletal muscle tissue, endometrial tissue, menstrual blood, and/or fallopian tube tissue.

36. The method of claim 35, wherein the fibroblasts are isolated from umbilical cord tissue.

37. The method of claim 36, wherein the fibroblasts:
(a) express oxidized low density lipoprotein receptor 1, chemokine receptor ligand 3, and/or granulocyte chemotactic protein;
(b) do not express CD117, CD31, CD34, and/or CD45;
(c) express increased levels of interleukin 8 and reticulon 1 relative to a human mesenchymal stem cell;
(d) are capable of differentiating into skeletal muscle cells, vascular smooth muscle cells, pericytes, vascular endothelial cells, osteocytes, adipocytes, and/or chondrocytes;
(e) express CD10, CD13, CD44, CD73, CD90, PDGFr-a, PD-L2, HLA-A, HLA-B, and/or HLA-C;
(f) do not express one or more of CD31, CD34, CD45, CD80, CD86, CD117, CD141, CD178, B7-H2, HLA-G, HLA-DR, HLA-DP, and/or HLA-DQ;
(g) secrete MCP-1, MIPIbeta, IL-6, IL-8, GCP-2, HGF, KGF, FGF, HB-EGF, BDNF, TPO, RANTES, and/or TIMP1; and/or
(h) express TRA1-60, TRA1-81, SSEA3, SSEA4, and/or NANOG.

38. (canceled)

39. (canceled)

40. (canceled)

41. (canceled)

42. (canceled)

43. (canceled)

44. (canceled)

45. The method of claim 1, further comprising performing a cellular graft in the subject, wherein the fibroblasts or fibroblast-derived products increase the quality of the cellular graft and/or further comprising providing a lithium-containing compound, or a pharmaceutically acceptable salt thereof, to the subject.

46. The method of claim 45, wherein the cellular graft is an islet transplant, a hepatocyte transplant, or a hematopoietic stem cell transplant.

47. (canceled)

48. The method of claim 45, wherein the lithium-containing compound is lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, or lithium succinate.

49. The method of claim 1, wherein the telomere-associated condition is dyskeratosis congenita, cancer, cellular senescence, idiopathic pulmonary fibrosis, Hoyeraal-Hreiderasson syndrome, Hutchinson-Gilford progeria, aplastic anemia, aging, or an age-related disease.

50. (canceled)

51. (canceled)

52. (canceled)
